(19) United States
(12) Patent Application Publication
(10) Pub. No.: US 2019/0327961 A1
(43) Pub. Date: Oct. 31, 2019

(54) STABLE SOLID COMPOSITION BASED ON AN AROMATIC COMPOUND AND USES THEREOF

(71) Applicant: Adnane REMMAL, FES (MA)

(72) Inventors: Adnane REMMAL, FES (MA); Mounia OUKHOUIA, FES (MA); Chaimae Imane SENNOUNI, FES (MA); Hakima MOUSSA, SEFROU (MA); Meryem MARZOUQ, FES (MA)

(21) Appl. No.: 16/462,123

(22) PCT Filed: Nov. 17, 2017
(86) PCT No.: PCT/IB2017/057198

§ 371 Date: May 17, 2019

(30) Foreign Application Priority Data
Nov. 18, 2016 (FR) 16/70691

(51) Int. Cl. A01N 25/14; A01N 3/02; A23B 7/154; A23L 3/349; A23L 3/3508; A61L 2/00; A61L 2/18; A61L 9/04; A01C 1/06; A01N 25/34

(52) U.S. Cl. CPC A01N 25/14; A01N 3/02; A23B 7/154; A23L 3/349; A23L 3/3508; A61L 2209/21; A61L 2/18; A61L 9/04; A01C 1/06; A01N 25/34; A61L 2202/24; A61L 2/0088

(57) ABSTRACT

Disclosed is a water-dispersible and stable solid composition including: at least one aromatic alcohol, at least one emulsifying agent and/or an oil; at least one texturing agent; and at least one effervescent acid-base pair. Also disclosed are methods for use of the composition, particular as a phytosanitary product.

### BACKGROUND OF THE INVENTION

### Field of the Invention

This invention relates to a stable composition based on a natural or synthetic aromatic compound as well as to its various uses, in particular in the agricultural field.

In the face of a steadily-increasing population, worldwide agriculture is confronted with multiple challenges, in particular the increased demand for foodstuffs. However, the plant and animal agricultural field is subject to multiple biotic problems, namely the illnesses caused by bacteria, viruses, fungi, nematodes, and all other insects and pests. To overcome these problems, most farmers and breeders use chemical products (pesticides and disinfectants) that are very harmful to human health and to the environment.

It is therefore urgent and necessary to find an alternative to these products that is both effective against the biotic attacks of crops and safe for human health and for the environment.

### Description of the Related Art

Multiple alternatives have recently been proposed for replacing the pesticides and the chemical disinfectants, in particular:
The use of microbial biopesticides (bacteria, fungi, or viruses), but these products have a high cost, cannot be industrialized on a large scale, and there are significant risks of mutation and development of resistance,
Resorting to varieties of resistant plants in using resistance genes, but these products are not satisfactory in particular because of their significant development time, the mediocre quality of the fruits obtained, and genes that are heavily influenced by the environment and are often poorly expressed,
Biopesticides based on plant extracts, but these products are no longer suitable due to both their liquid form that makes storage quite difficult since it requires a precise temperature and humidity, as well as the risk of evaporation of the active ingredients.

Among the biopesticides based on plant extracts, several studies have focused on essential oils and their majority components, such as thymol, carvacrol, eugenol in particular (Koul, O. et al. (2008). Essential Oils as Green Pesticides: Potential and Constraints. Biopestic. Int. 4(1): 63-84; Isman, M. B. and Machial, C. M. (2006). Pesticides Based on Plant Essential Oils: From Traditional Practice to Commercialization. In M. Rai and M. C. Carpinella (Eds.), Naturally Occurring Bioactive Compounds, Elsevier, BV, pp. 29-44). These compounds actually have very promising properties. However, they present problems linked to their volatility, their poor solubility in water, and their capacity for being oxidized quickly (Moretti, M. D. L. et al. (2002). Essential Oil Formulations Useful as a New Tool for Insect Pest Control. AAPS Pharm. Sci. Tech. 3 (2): 13). The volatility of these compounds is at the origin of their short service life in solution. This negatively affects the quality of the formulation since the manufacturers have to use large amounts of it or have to use sophisticated and expensive stabilization methods such as micro- and nano-encapsulation (WO2007063267A1), which makes these products non-competitive in relation to chemical pesticides.

### SUMMARY OF THE INVENTION

The object of this invention is therefore to find a solution to these problems and to propose in particular a stable formulation comprising aromatic compounds of a volatile nature having an antimicrobial activity (antibacterial, antifungal, antiviral, and antiparasitic) and/or anti-insect.

To respond to this, the invention has as its object a composition in a form that is solid, stable, and dispersible in water, comprising:
At least one aromatic alcohol or at least one mixture containing it, such as an essential oil,
At least one emulsifying agent and/or one oil,
At least one texturing agent, and
At least one effervescent acid-base pair.

Advantageously, such a composition:
Is stable, whereas it comprises molecules, with volatile origins,
Comes in solid form, which facilitates its storage, and
Is dispersible in water, which makes possible its use mixed with water before use.

The aromatic alcohols have numerous properties depending on their chemical formula, and the composition according to the invention can advantageously be used in particular as a phytosanitary product or fertilizer, but the composition according to the invention can also be used for other applications, such as, for example, an anti-insect application or as an antimicrobial application for sanitizing ambient air, hands, buildings, reusable medical devices, etc.

### BRIEF DESCRIPTION OF THE DRAWINGS

Other characteristics and advantages of the invention will emerge from the description of the invention, in details, which will follow, and test results produced with the invention demonstrating its effectiveness in numerous applications, results illustrated in particular by the accompanying figures that show:

FIG. 1: an image of the composition according to the invention of Example 1, before dispersion,

FIG. 2: an image of the C3 composition compared to the composition according to the invention in Table 1,

FIG. 3: an image of the C4 composition compared to the composition according to the invention in Table 1.

### DESCRIPTION OF THE PREFERRED EMBODIMENTS

### Definitions

In terms of the invention, “emulsifying agent” is defined as any compound that can improve the suspension and the dispersion of a composition in water.

When the term emulsifying agent is used in the singular in this application, one or more emulsifying agent(s) should be understood, with the composition according to the invention being able to comprise one or more emulsifying agent(s).

In terms of the invention, “texturing agent” is defined as any compound that has the functions of stabilizing (improves the stability), thickening (increases the viscosity), and/or emulsifying (improves the suspension).

When the term texturing agent is used in the singular in this application, one or more texturing agent(s) should be understood, with the composition according to the invention being able to comprise one or more texturing agent(s).

In terms of the invention, “aromatic alcohol” is defined as an aromatic molecule, having a hydroxyl group OH that is attached to a carbon of a benzene cycle. The aromatic alcohol is also called a phenol.

The term “aromatic alcohol” can be used interchangeably in the singular or in the plural in this application. Whether it is used in the singular or in the plural, at least one aromatic alcohol, i.e., one or more aromatic alcohol(s), should always be understood, with the composition according to the invention being able to comprise one or more aromatic alcohol(s).

In terms of the invention, “anti-insect” is defined as any inhibiting and/or destructive action of insects and pests.

In terms of the invention, “antimicrobial” is defined as any inhibiting and/or destructive action of germs that are bacterial, fungal, parasitic, and viral.

In terms of the invention, “compound” is defined as a molecule or a mixture of molecules.

In terms of the invention, “effervescent acid-base pair” is defined as the association, the combination, of an acid and a base that can produce an effervescent phenomenon when said pair is put into solution.

When the term effervescent acid-base pair is used in the singular in this application, one or more acid-base pair(s) should be understood, with the composition according to the invention being able to comprise one or more acid-base pair(s).

In terms of the invention, “dispersible in water” is defined as being able to disperse, to solubilize in water without forming non-dispersible masses, in particular in irrigation water for spraying plants or watering animals.

In terms of the invention, “essential oil” is defined as any extract that is obtained from one or more aromatic plant(s), preferably the concentrated and hydrophobic liquid of volatile aromatic (fragrant) compounds of a plant. An essential oil can be obtained in particular by mechanical extraction and vapor entrainment or by dry distillation. “Essential oil” is also defined as products that are identical to those described above but obtained by chemical synthesis.

In terms of the invention, “mixture containing an aromatic alcohol” is defined as any mixture of molecules comprising at least one aromatic alcohol. It may preferably involve one or more essential oil(s) or one or more mixture(s) of molecules contained in one or more essential oil(s) or a mixture of one or more essential oil(s) with one or more mixture(s) of molecules contained in one or more essential oil(s).

When the term mixture containing an aromatic alcohol is used in the singular in this application, one or more mixture(s) containing an aromatic alcohol should be understood, with the composition according to the invention being able to comprise one or more mixture(s) containing an aromatic alcohol.

In terms of the invention, “stable” product is defined as a product whose molecular composition as well as the texture, color, and effectiveness are constant over time.

In terms of the invention, “solid” is defined as a preferably uniform non-liquid form: powder, pellet, or tablet.

### DETAILED DESCRIPTION OF THE INVENTION

The invention therefore has as its object a composition in a form that is solid, stable, and dispersible in water.

The composition according to the invention can in particular come in the form of powder, pellets, or tablets.

The composition according to the invention comprises at least one aromatic alcohol or at least one mixture containing at least one aromatic alcohol.

Said at least one aromatic alcohol is a natural or synthetic aromatic alcohol.

Preferably, said at least one aromatic alcohol is mono-, di- or sesquiterpenic. It can be selected in particular from among thymol, menthol, eugenol, carvacrol, and cinnamaldehyde.

According to a particularly suitable embodiment, the composition comprises at least one aromatic alcohol that is contained in an essential oil, and preferably the composition according to the invention comprises at least one essential oil, such as, for example, the essential oil of thyme, oregano, clove, mint.

Said at least one aromatic alcohol and/or said at least one mixture comprising at least one aromatic alcohol, present in the composition according to the invention, play(s) an active ingredient role. They have effects and effectiveness that vary depending on their nature, but they preferably have an antibacterial and/or anti-insect activity, as is the case in particular of aromatic alcohols selected from among thymol, menthol, eugenol, carvacrol, and cinnamaldehyde.

In a preferred way, the aromatic alcohol or the mixture comprising at least one aromatic alcohol represents between 0.1 and 25% by weight of the composition.

In addition to the aromatic alcohol (or the mixture containing it), the composition according to the invention also comprises at least one emulsifying agent and/or one oil. The emulsifying agent and/or oil is/are preferably selected from among vegetable oils, mineral oils, soy lecithin, cellulose, pectin, and glycerol.

Said at least one emulsifying agent and/or oil makes it possible in particular to liquefy and/or to keep in suspension the aromatic alcohol when it is put into solution. Actually, the emulsifiers and/or oils interact both with the aromatic alcohol molecules and with the water molecules, which prevents the aromatic alcohol molecules from floating to the surface of the water.

In a preferred way, the emulsifying agent and/or the oil represent(s) between 0.01 and 2% by weight of the composition.

The composition according to the invention, in addition to these first two components, also comprises at least one texturing agent. It is preferably selected from among locust bean gum, guar gum, and cassia gum.

Said at least one texturing agent makes it possible in particular to improve the stabilization of the aromatic alcohols present in the composition both during storage and during the solubilization. Actually, the texturing agent makes it possible, during manufacturing, to fix the aromatic alcohols and the emulsifiers and/or oils that are liquid and consequently to obtain a sievable powder. During storage, the volatile aromatic alcohols remain fixed on the texturing agents and do not evaporate. At the time of the solubilization, the texturing agents create a viscosity that promotes the dispersion of aromatic alcohols and prevent them from floating.

In a preferred way, said at least one texturing agent represents between 5 and 12% by weight of the composition. The ratio of the gum does not exceed 12%, preferably 10%, in the final composition, if not once in solution, the texturing agent runs the risk of forming a paste that is difficult to disperse.

Finally, the composition according to the invention also comprises at least one effervescent acid-base pair. It is preferably selected from among the following acid-base pairs: sodium bicarbonate-citric acid, calcium bicarbonate-citric acid, potassium bicarbonate-citric acid, sodium bicarbonate-tartaric acid, calcium bicarbonate-tartaric acid, potassium bicarbonate-tartaric acid, sodium bicarbonate-maleic acid, potassium bicarbonate-maleic acid, sodium bicarbonate-ascorbic acid, calcium bicarbonate-ascorbic acid, and potassium bicarbonate-ascorbic acid. Preferably, the acid from the effervescent pair represents between 20 and 25% by weight of the composition, and the base of the effervescent pair represents between 60 and 74.89% by weight of the composition.

Said at least one effervescent acid-base pair that is present in the composition makes it possible in particular to improve the dissolution of the aromatic alcohol once in solution.

In a preferred way, said at least one effervescent acid-base pair represents between 50 and 94.89% by weight of the composition.

According to a variant, the composition according to the invention consists exclusively of:
One or more aromatic alcohol(s) or one or more mixture(s) containing one or more aromatic alcohol(s),
One or more emulsifying agent(s) and/or one or more oil(s),
One or more texturing agent(s), and
One or more effervescent acid-base pair(s).

One composition according to this variant can consist in particular as follows:
The aromatic alcohol(s) or the mixture(s) of aromatic alcohol(s) represent(s) between 0.1 and 25% by weight of the composition,
The emulsifying agent(s) and/or the oil(s) represent(s) between 0.01 and 2% by weight of the composition,
The texturing agent(s) represent(s) between 5 and 12% by weight of the composition,
The effervescent acid-base pair(s) represent(s) between 61 and 94.89% by weight of the composition.

According to another variant, in addition to said at least one aromatic alcohol (or at least one mixture containing at least one aromatic alcohol), said at least one emulsifying agent and/or oil, said at least one texturing agent, and said at least one effervescent acid-base pair, the composition according to the invention comprises at least one other compound, such as, for example, at least one compound that is selected from among the vitamins, minerals, hormones, dyes, etc.

The composition according to the invention can be obtained by any suitable method that makes it possible to obtain a composition in solid form with the above-mentioned components.

It can involve in particular a method comprising the following steps:
At least one aromatic alcohol or at least one mixture containing at least one aromatic alcohol, with said alcohol or said mixture being in liquid or crystallized form, is mixed with at least one emulsifying agent and/or one oil until a clear solution is obtained, preferably under the following conditions:
   While being stirred, in particular in a tank with a stirring mechanism rotating between 30 and 120 rpm, preferably for 5 to 45 minutes,
   At a temperature of between 40 and 60° C., and/or
   Without light, in darkness.

This first step makes it possible to obtain good dissolution of the aromatic alcohols, without any recrystallization problem.

The solution is then added to a powder containing at least one texturing agent and an effervescent acid-base pair, preferably under the following conditions:
While being stirred, in particular in a mixer/mixing mechanism, even more preferably for 10 to 40 minutes, and/or
At ambient temperature, and/or
Without light, in darkness.

The texturing agent makes it possible to stabilize the volatile molecules by absorption in the final composition. The effervescent acid-base pair improves the solubilization of the composition that can be carried out without necessarily having recourse to manual or mechanical stirring.

During the stirring in the mechanical mixing mechanism, the initial powder that consists of the texturing agent and the acid-base pair gradually absorbs the liquid that consists of the aromatic alcohol and the emulsifying agent and/or oil until a powder sometimes containing small lumps is obtained. These small lumps are converted into powder by the action of the calibrator; the calibration is done after the powder exits from the mixing mechanism. (The final powder obtained after calibration is preferably stored in hermetically-sealed bags protected from heat and humidity at ambient temperature.) Depending on the aromatic alcohols, and/or mixtures containing them, present in the composition, the composition according to the invention can be used for various applications. Before use, the solid composition according to the invention is preferably put into solution. In a preferred way, the composition/water ratio is between 100 g per 1,000 liters of water up to 25 kg per 1,000 liters of water.

In particular, the aromatic alcohols have antibacterial and/or antifungal and/or antiviral and/or anti-insect properties.

The object of the invention is therefore in particular the use of a composition according to the invention as a phytosanitary product, in particular for preventing and/or combatting the plant illnesses (some or all of the plants) caused by fungi, bacteria, viruses, nematodes and/or pests.

For its use by application on the plants (some or all of the plants), including on fruits, vegetables and/or flowers post-harvest, the composition according to the invention can be used as irrigation water or in leaf spraying.

The plant illnesses caused by bacteria for which the composition according to the invention is particularly useful for prevention and/or treatment are preferably selected from among the soft rot caused by the Erwinia species, the bacterial canker caused by the Pseudomonas species, or the crown gall caused by the Agrobacterium species.

The plant illnesses caused by fungi for which the composition according to the invention is particularly useful for prevention and/or treatment are preferably selected from among the Fusarium wilt caused by the Fusarium species, the mildew caused by the Phytophtora species, the powdery mildew caused by the Podosphaera and Oidium species, the early blight caused by the Alternaria species, the sooty mold caused by the Alternaria and Cladosporium species, or the gray rot caused by the Botrytis species.

The pests causing plant illnesses against which the composition according to the invention is particularly useful for prevention and/or treatment are selected from among aphids, gnats, soil mites, or budworms.

The composition according to the invention can be used specifically for preventing and/or combatting the illnesses of fruits, vegetables, and/or flowers post-harvest caused by fungi, bacteria, viruses, nematodes, and/or pests. In particular, the composition according to the invention can be used for preventing and/or combatting the rot of citrus fruit caused by the Penicillium and Geotrichum species, or for preventing and/or combatting the fungal deterioration of dates caused by the Aspergillus species.

The composition according to the invention can also be used for an antifungal treatment during the coating of seeds, in particular seeds selected from among wheat, barley, lentils, chickpeas, and beans.

According to another aspect, the composition according to the invention can be used as a phytosanitary product for:
Increasing the vase life of cut flowers, or
Cleaning and eliminating the traces of pesticides and microbes contained in fruits and vegetables, in particular for decontaminating grapes, apricots, and peppers before the drying.

The composition according to the invention can also advantageously be used to stimulate the growth of plants, in particular to stimulate the rhizogenesis in plants.

Independently of its use in application on the vegetables, either as a phytosanitary product or for stimulating their growth, the composition according to the invention can be used directly on objects, soils, walls, etc., in particular for:
Decontaminating agricultural farming substrates above ground and in the ground (for example, sand, topsoil, and perlite) and/or sanitizing the ambient air, in particular of industrial buildings,
Cleaning and preventing the formation of limestone, in particular in drippers,
In the animal-breeding buildings, for example in the poultry-breeding buildings, in particular:
   Sanitizing the ambient air of buildings, and/or
   Decontaminating drinking water, and/or
   Cleaning the surfaces of the buildings,
Eliminating the biofilms and preventing their formation, in particular in the water supply pipes in the animal-breeding buildings, for example in the poultry-breeding buildings,
In the poultry-breeding buildings, for preventing and/or treating avian flu, by application to the building, by eliminating the viruses that are present on the walls and the floors of the buildings.

The composition according to the invention can also be used as a bath for decontaminating animal carcasses in slaughterhouses or for decontaminating poultry eggs before placing them in incubators.

According to another aspect, the composition according to the invention is particularly effective and can be used to disinfect reusable medical devices, in particular fibroscopes, coloscopes, bronchoscopes, and sinuscopes.

The composition can also be used:
As a disinfectant mouthwash for teeth and gums, or
As a disinfectant product for hands, in particular for hospital and medical personnel.

The composition according to the invention can therefore be used for numerous applications. It offers the advantage of coming in solid and stable form, which makes possible a long-term storage of at least two years and an easy and economical transfer. In addition, the composition is very easily dispersible in water, which makes possible an easy and economical use in solution.

The invention is now illustrated by non-limiting examples of compositions according to the invention, of uses and test results demonstrating their effectiveness.

### Example 1

The composition of Example 1 consists of:
15% thymol
0.2% soy lecithin
10% locust bean gum
50.54% sodium bicarbonate
24.26% citric acid.

This composition is obtained by implementing the following steps:

15 g of thymol is preheated to 50° C. and mixed with 0.2 ml of soy lecithin, and then the mixture is added to the combination of 10 g of locust bean gum, 50.54 g of sodium bicarbonate, and 24.26 g of citric acid. All of it is suspended in 10 l of water.

### Example 2

The composition of Example 2 consists of:
15% menthol
0.2% mineral oil
10% guar gum
50.54% potassium bicarbonate
24.26% tartaric acid.

This composition is obtained by implementing the following steps:

15 g of menthol is preheated to 50° C. and mixed with 0.2 ml of mineral oil, and then the mixture is added to the combination of 10 g of guar gum, 50.54 g of potassium bicarbonate, and 24.26 g of tartaric acid. All of it is suspended in 10 l of water.

### Example 3

The composition of Example 3 consists of:
15% carvacrol
0.2% glycerol
10% xanthan gum
50.54% potassium bicarbonate
24.26% maleic acid.

This composition is obtained by implementing the following steps:

15 ml of carvacrol is preheated to 50° C. and mixed with 0.2 ml of glycerol, and then the mixture is added to the combination of 10 g of xanthan gum, 50.54 g of potassium bicarbonate, and 24.26 g of maleic acid. All of it is suspended in 10 l of water.

### Example 4

The composition of Example 4 consists of:
15% eugenol
0.2% soy lecithin
10% locust bean gum
50.54% sodium bicarbonate
24.26% tartaric acid

This composition is obtained by implementing the following steps:

15 ml of menthol is preheated to 50° C. and mixed with 0.2 ml of soy lecithin, and then the mixture is added to the combination of 10 g of locust bean gum, 50.54 g of sodium bicarbonate, and 24.26 g of tartaric acid. All of it is suspended in 10 l of water.

### Comparison Tests Demonstrating the Need for the Combination of Various Components of the Composition for Making the Composition Stable, Homogeneous, and Dispersible in Water

The aromatic alcohols do not disperse easily in water because of their hydrophobic nature. The prior art proposes a method for dispersion of these compounds by the addition of a surfactant and a solvent (WO2009124392A1), but this method is not suitable because it is known that the surfactants and the solvents reduce the activity of the phenolic compounds (Remmal, A., Bouchikhi, T., Tantaoui-Elaraki, A., Ettayebi, M. (1993). Inhibition of Antibacterial Activity of Essential Oils by Tween 80 and Ethanol in Liquid Medium. J. Pharm. Belg. 48: 352-356). In addition, certain aromatic alcohols, such as thymol or menthol, are in crystallized form at ambient temperature, and once in contact with water, if an attempt is made to make them liquid by heating them, they recrystallize.

The composition according to the invention combining the aromatic alcohols with a texturing agent, an emulsifying agent (and/or one oil), and an effervescent acid-base pair makes it possible at the same time to prevent the recrystallization of aromatic alcohols in crystallized form, to ensure the stability of aromatic alcohols and of the composition, its homogeneity, and to facilitate its dispersion during its solubilization.

The form and the dispersion in water of the composition of Example 1 was evaluated in comparison to other compositions comprising just an aromatic alcohol or with only one or more component(s) of the composition according to the invention.

### Composition C1

The composition C1 consists of 15% thymol only.

This composition is obtained by the suspension of 15 g of thymol in 10 l of water.

### Composition C2

The composition C2 that consists of:
15% thymol
0.2% soy lecithin

This composition is obtained by implementing the following steps:
15 g of thymol is preheated to 50° C. and mixed with 0.2 ml of soy lecithin,
All of it is suspended in 10 l of water.

### Composition C3

The composition C3 that consists of:
15% thymol
0.2% soy lecithin
56.54% sodium bicarbonate
28.26% citric acid

This composition is obtained by implementing the following steps:
15 g of thymol is preheated to 50° C. and mixed with 0.2 ml of soy lecithin,
Then, the mixture is added to the combination of sodium bicarbonate and citric acid,
All of it is suspended in 10 l of water.

### Composition C4

The composition C4 that consists of:
15% thymol
0.2% soy lecithin
84.8% locust bean gum

This composition is obtained by implementing the following steps:
15 g of thymol is preheated to 50° C. and mixed with 0.2 ml of soy lecithin,
Then the mixture is added to 84.8 g of locust bean gum,
All of it is suspended in 10 l of water.

The results are presented in Table 1 and in the figures.

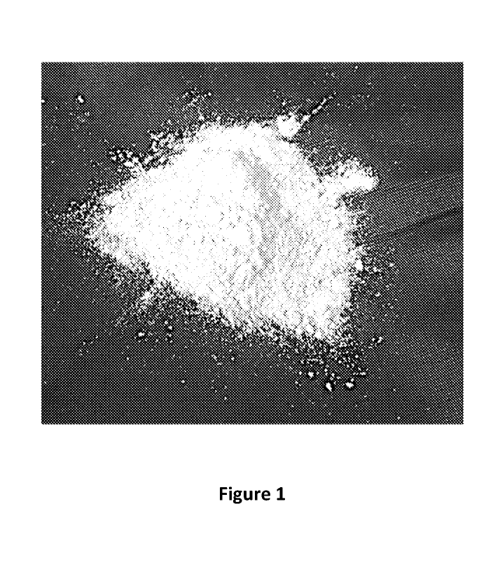

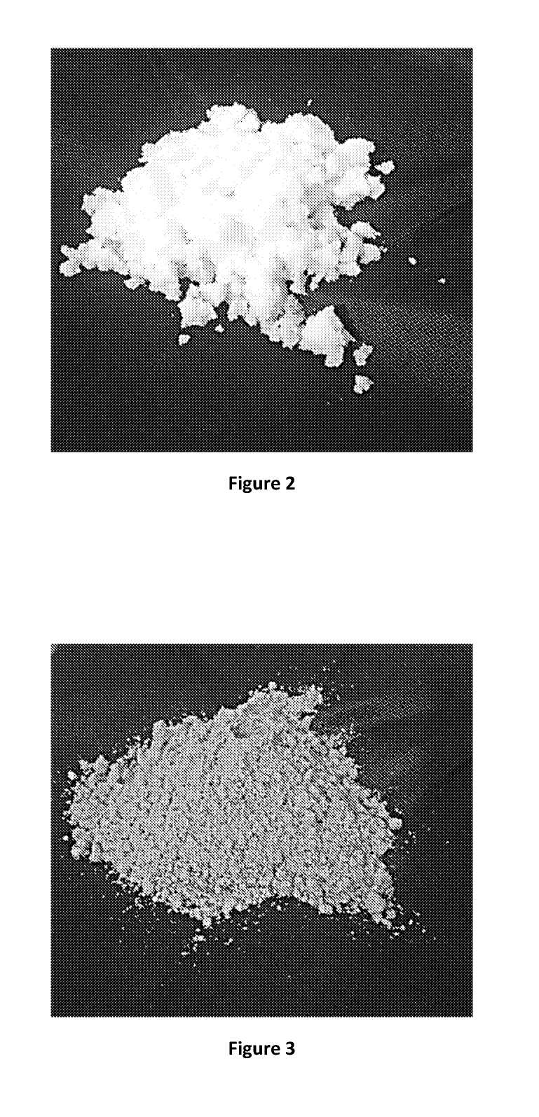

TABLE 1
Results of the Various Formulation Attempts

| Com- | | Dispersion in | |
|---|---|---|---|
| position | Form | Water | Results |
| C1 | Crystallized | Non-dispersible | Presence of thymol |
| | | | crystals on the |
| | | | surface |
| C2 | Liquid | Non-dispersible | Presence of an oily |
| | | | layer on the surface |
| C3 | Unusable whitish | NA | NA |
| | moist powder | | |
| | (non-sievable | | |
| | powder) | | |
| | FIG. 2 | | |
| C4 | Beige-colored | Sparingly dispersible | NA |
| | moist powder | (requires manual | |
| | FIG. 3 | stirring) formation of | |
| | | a foot. Unusable | |
| Example 1 | Light beige- | Easily dispersible | Easy and total |
| (Invention) | colored | | dispersion in water |
| | homogeneous | | without the need for |
| | powder. Sievable | | stirring |
| | FIG. 1 | | |

NA: Not Applicable

These results reveal that only the combination of the components of the composition according to the invention makes it possible to obtain a powder containing an aromatic alcohol that is stable, homogeneous, and easily dispersible.

### In Vitro Test of the Antimicrobial Activity of the Composition According to the Invention

For the purpose of evaluating the effectiveness of the composition according to the invention (Example 1): it was tested in vitro on several strains of bacteria, yeasts, and molds causing illnesses and damage in plants.

The 15 strains tested in this study were isolated, purified, and identified: 11 strains of fungal origin, including 5 of the genus Fusarium, one of the genus Penicillium, one of the genus Geotrichum, one of the genus Alternaria, one of the genus Sclerotonia, and the last of the genus Helminthosporium. Two strains of yeast origin, one of the genus Candida, and the other of the genus Saccharomyces. The two strains that remain are of bacterial origin.

The culture media used are:
Sabouraud dextrose agar and broth (Biokar) for the cultivation and testing of the antifungal activity of the fungal strains.
Mueller-Hinton agar and broth (Biokar) for the cultivation and testing of the antibacterial activity of the bacterial strains.

The testing of the antifungal activity of the composition according to the invention was carried out as follows:
The culture media were prepared according to the instructions of the supplier; various concentrations of the composition were used for the purpose of determining the CMI (minimal inhibiting concentration) in a gelose medium and in a liquid medium, the CMF (minimal fungicidal concentration) in a liquid medium by using the macrodilution technique.
In gelose medium: In flasks containing 60 ml of the appropriate medium, sterilized in the autoclave for 20 minutes at 121° C. and cooled to 45° C., various quantities of the composition of Example 1 are added, making it possible to obtain the following final concentrations: (0.41; 0.82; 1.65; 3.33; 6.67; 13.34; 26.68, and 53.36 g/l). Negative controls containing just the medium are also prepared; the flasks are then poured into Petri dishes (90×16 mm) and allowed to cool to 4° C. for 24 hours, then inoculated with a volume of 10 μl of an initial suspension of 10⁶ spores/ml of the appropriate mold. After diffusion of the deposited drop, the dishes are incubated at 27° C. for 5 days. The test is performed in triplicate and is repeated three times.
In liquid medium: the Sabouraud culture broth that is sterilized in the autoclave for 20 minutes at 121° C. and cooled to ambient temperature was distributed in sterile test tubes. Then, increasing concentrations of the composition according to the invention of: (0.41; 0.82; 1.65; 3.33; 6.67; 13.34; 26.68, and 53.36 g/l) were tested. All of the tubes were inoculated with 20 μl of the initial suspension of spores (10⁶ spores/ml). The final concentration in each tube is calculated for a final volume of 5 ml. The tubes are thus incubated at 27° C. with stirring at 130 rpm for 5 days.
Determination of the CMF (Comparative Mortality Figure): Starting from wells where there has not been visible positive growth, a fraction of 20 μl is aseptically sampled and transferred into Eppendorf tubes containing 1 ml of Sabouraud broth. Thus, the risk of transferring the inhibiting effect of the composition is eliminated by a 50× dilution. The CMF of each composition is defined as being the smallest concentration of the composition for which there is a total absence of any fungal growth in comparison with the control.

The testing of the antibacterial activity of the composition according to the invention was carried out by following the same approach as the one produced in the testing of the antifungal activity except that the culture medium used for this test is Mueller-Hinton (agar and broth); the inoculated bacterial inoculum is on the order of 10⁷ bacteria/ml, and the incubation temperature is on the order of 37° C.

The values of CMI and CMF obtained with the composition according to the invention are summarized in Table 2.

TABLE 2
CMI and CMF Values of the Composition According to the
Invention for the Fungal Species

| | Example 1 Composition | |
|---|---|---|
| | CMI (g/1) | CMF (g/1) |
| Fusarium oxysporum spdianthi | 1.65 | 3.33 |
| Fusarium oxysporum spalbedinis | 0.82 | 1.65 |
| Fusarium oxysporum spgladioli | 1.65 | 3.33 |
| Fusarium oxysporum spcubense | 1.65 | 3.33 |
| Fusarium nival | 0.82 | 1.65 |
| Penicillium digitatum | 1.65 | 3.33 |
| Geotrichum candidum | 0.82 | 1.65 |
| Alternaria alternata | 1.65 | — |
| Sclerotinia homoeocarpa | 1.65 | 3.33 |
| Helminthosporium | 1.65 | 3.33 |
| Cladosporium | 3.33 | — |
| Candida albicans | 1.65 | 3.33 |
| Saccharomyces cerevisiae | 1.65 | 3.33 |

According to these results, the composition has proven very effective against the fungal species used in this testing.

In addition, the CMI and CMB values obtained with the composition of Example 1 in the testing of the antifungal activity on the species of bacteria are presented in Table 3.

TABLE 3
CMI and CMB Values of the Composition According to the
Invention for the Species of Bacteria

| | Composition According to | |
|---|---|---|
| | the Invention | |
| | CMI (g/l) | CMB (g/l) |
| Escherichia coli | 1.65 | 3.33 |
| Staphylococcus aureus | 1.65 | 3.33 |

These results reveal a very significant antibacterial activity of the composition according to the invention.

### Testing of the Anti-Insect Activity of the Composition According to the Invention

For the purpose of evaluating the effectiveness of the composition according to the invention of Example 1, it was tested in vitro or in vivo on several insects, parasites, and nematodes.

The adults of the Macrosiphum rosae species (rosebush aphid) were sampled using a fine brush and brought into contact with various doses of the composition according to the invention.

In the Petri dishes, we deposit an absorbent paper disk saturated with solutions containing increasing doses of the composition according to the invention (1.67; 3.33; 6.67, and 13.34 g/l). Petri dishes were used as controls containing regular water. 20 adults were put into each dish; the experiment was set up in triplicate.

Likewise, the in vivo effectiveness of the composition according to the invention (Example 1) was tested on the Rhizoglyphus callae species (soil mite) in their usual environment, which is the soil. To do this, flower pots (freesia) containing soil naturally infested with these creatures (brought in from a farm producing cut flowers) were irrigated with solutions containing increasing doses of the composition according to the invention (1.67; 3.33; 6.67, and 13.34 g/l). Pots being used as controls received only water as an irrigation solution; the experiment was repeated three times.

After several hours of contact with the irrigation solution, several grams of soil from each pot were sampled and suspended in water; observation and counting under the binocular magnifying glass were carried out for the purpose of determining the percentage of mortality for each dose used in this testing.

In addition, the composition according to the invention was also tested in vitro on nematodes and more specifically the Heterodera spp species starting from infected soil (previously treated with various concentrations of the composition of Example 1). The method used is that of Baerrman. It is a method that consists in separating the nematodes from soil particles depending on their sizes and their weights according to the following steps:
Taking a 100 g soil aliquot, then passing it through a sieve, whose meshes are 2 mm in diameter, under a stream of water, so as to eliminate all of the large particles (gravel).
Recovering the suspension in ajar, then stirring to homogenize the contents (muddy solution), decanting for several minutes, then pouring the suspension containing the nematodes onto a sieve.
Recovering in a 50-ml beaker the suspension containing the nematodes and passing the contents into a funnel, on which a filter paper is deposited. After 24 hours, collecting the nematodes in a 10-ml volume of suspension and then counting under the binocular magnifying glass.
The thus counted nematodes are brought into contact with various doses of the composition according to the invention.

The dilutions that are used are:

1.67 g of the composition in 1 l of water

3.33 g of the composition in 1 l of water

6.67 g of the composition in 1 l of water

13.34 g of the composition in 1 l of water.

The Application Mode by Spraying

The mortality percentage of the insects used in this testing was calculated according to the following formula:

$$M ( \% ) = \frac{Nt - Nm}{Dt} \times 100$$

where Nt and Nm respectively represent the total number of insects and the number of dead insects.

The results are presented in Table 4.

TABLE 4
Mortality Percentage of Multiple Insects in Contact with the
Composition According to the Invention

| | Mortality in % | | | | |
|---|---|---|---|---|---|
| | 0 g/l | 1.67 g/l | 3.33 g/l | 6.67 g/l | 13.34 g/l |
| Macrosiphum | 0 ± 0.0 | 35 ± 3.3 | 70 ± 3.3 | 100 ± 0.0 | 100 ± 0.0 |
| rosae | | | | | |
| (rosebush aphid) | | | | | |
| Rhizoglyphus | 0 ± 0.0 | 56.6 ± 5.5 | 100 ± 0.0 | 100 ± 0.0 | 100 ± 0.0 |
| callae | | | | | |
| (soil mite) | | | | | |
| Heterodera spp | 0 ± 0.0 | 43.3 ± 4.4 | 73.3 ± 4.4 | 100 ± 0.0 | 100 ± 0.0 |
| (nematode) | | | | | |

These results clearly reveal that the composition according to the invention generates a very powerful insecticide effect. The 6.67 g/l dose has proven mortal for aphids and nematodes while the 3.33 g/l dose is effective against soil mites, after several hours of contact only.

### In Vivo Testing of the Antifungal Activity of the Composition According to the Invention

The composition according to the invention (Example 1) was tested in vivo on the vascular Fusarium wilt in carnations caused by Fusarium oxysporum sp dianthi. Eight seedlings per batch of the Martina variety were used in this testing.

The fusarium spores (10⁶ spores/ml) were inoculated into the siliceous sand near the roots of each plant.

Five batches were set up:

PNINT: Untreated, non-infected plants.

PINT: Untreated, infected plants.

PIT 3.33 g/l: Infected plants treated with 3.3 g/l of the composition according to the invention.

PIT 6.67 g/l: Infected plants treated with 6.67 g/l of the composition according to the invention.

PIT 13.34 g/l: Infected plants treated with 13.34 g/l of the composition according to the invention.

To evaluate the effectiveness of the composition, the measurements of multiple agromorphological types were compared, in particular the number of shoots, the distance between nodes, the number of nodes, the length of the stem, the diameter of the stem, and the number of flower buds.

The dilutions used are:

3.33 g of the composition in 1 l of water.

6.67 g of the composition in 1 l of water.

13.34 g of the composition in 1 l of water.

The application mode is an application by irrigation.

The results that are obtained are presented in Table 6.

TABLE 6
Effect of the Composition According to the Invention on
the Growth of Carnations Infected by Fusarium Wilt

| | | | | PIT | |
|---|---|---|---|---|---|
| | | PIT 3.33 | PIT 6.67 | 13.34 | |
| Martina | PINT | g/l | g/l | g/l | PNINT |
| Number of | 3.12 ± 0.43 | 3.62 ± 0.46 | 3.4 ± 0.48 | — | 3.87 ± 0.46 |
| Shoots | | | | | |
| Distance | 4.12 ± 0.40 | 5.18 ± 0.40 | 5.1 ± 0.16 | — | 3.75 ± 0.43 |
| Between the | | | | | |
| Nodes | | | | | |
| Number of | 10.12 ± 1.12 | 14 ± 1 | 14.4 ± 0.48 | — | 13.87 ± 1.15 |
| Nodes | | | | | |
| Length of the | 58.5 ± 5 | 80 ± 5 | 74.6 ± 2.88 | — | 66.3 ± 3.9 |
| Stem | | | | | |
| Diameter of | 5.42 ± 0.25 | 8.22 ± 0.5 | 7.34 ± 0.64 | — | 6.09 ± 0.57 |
| the Stem | | | | | |
| Number of | 0 ± 0.00 | 9.75 ± 1.3 | 8.6 ± 1.76 | — | 4.12 ± 2.84 |
| Flower Buds | | | | | |

These results clearly reveal that the plants that are infected and treated by the composition according to the invention do much better than the non-infected plants; the dose that provided the better results in terms of agromophological types is 3.3 g/l.

Furthermore, the dose 13.34 g/l has proven to be too high for the carnations, since it generated a phytotoxic effect on the aerial part and even on the root system of the plant.

The importance of these results comes from the fact that the fungal load of the soil is a decisive factor for the health of the plant; actually, the higher this load is, the more the plant is stressed by the bacteria and the fungi that enter into competition with the plant on the essential nutrients for its growth.

The use of the composition in the irrigation water therefore makes it possible to reduce the fungal load of the soil. This will have a positive impact on the health and well-being of the young plant that develops.

### Stimulating Effect of the Growth of the Composition According to the Invention

The stimulating effect of the growth of the composition according to the invention (Example 1) was also tested on seedlings of carnations of West Diamond variety.

To do this, five batches were set up:

PNT: Non-treated plants.

PT 1 g/l: Plants treated with 1 g/l of the composition according to the invention.

PT 1.65 g/l: Plants treated with 1.65 g/l of the composition according to the invention.

PT 3.33 g/l: Plants treated with 3.33 g/l of the composition according to the invention.

PT 6.67 g/l: Treated with 6.67 g/l of the composition according to the invention.

The stimulating effect of the composition according to the invention (Example 1) was quantified by taking measurements of the agromorphological types (the number of shoots, the distance between nodes, the number of nodes, the length of the stem, the diameter of the stem, and the number of flower buds) after three months of treatment at a rate of three applications per week of irrigation water.

The dilutions used are:

1 g of the composition in 1 l of water.

1.67 g of the composition in 1 l of water.

3.3 g of the composition in 1 l of water.

6.67 g of the composition in 1 l of water.

The application method is by irrigation.

The results that are obtained are presented in Table 7.

TABLE 7
Stimulating Effect of the Growth of the Composition According to
the Invention on Carnation Seedlings

| West | | | | | |
|---|---|---|---|---|---|
| Diamond | PNT | PT 1 g/l | PT 1.65 g/l | PT 3.3 g/l | PT 6.67 g/l |
| Number of | 2.75 ± 0.56 | 3 ± 0.25 | 2.85 ± 0.77 | 3.25 ± 0.56 | 3.42 ± 0.48 |
| Shoots | | | | | |
| Distance | 2.93 ± 0.34 | 3.68 ± 0.64 | 3.5 ± 0.57 | 3.5 ± 0.77 | 3.35 ± 0.59 |
| Between the | | | | | |
| Nodes | | | | | |
| Number of | 12.75 ± 1.06 | 13 ± 1.25 | 13.28 ± 0.61 | 13.75 ± 0.81 | 13 ± 1.14 |
| Nodes | | | | | |
| Length of the | 49.25 ± 6.43 | 52.25 ± 4.25 | 51.57 ± 3.34 | 57 ± 2.75 | 53.85 ± 3.26 |
| Stem | | | | | |
| Diameter of | 6.56 ± 0.19 | 7.00 ± 0.41 | 7.71 ± 0.33 | 7.83 ± 0.46 | 7.54 ± 0.26 |
| the Stem | | | | | |
| Number of | 1.75 ± 1.31 | 2.87 ± 1.65 | 2.42 ± 1.51 | 4.12 ± 1.40 | 2.85 ± 1.63 |
| Flower Buds | | | | | |

These results reveal a convincing effect of the composition according to the invention; the dose 3.3 g/l has proven most effective since the plants that have received this dose of irrigation water have very high-performing agromorphological natures compared to the control plants.

### Stimulating Effect on Rhizogenesis of the Composition According to the Invention

The stimulating effect of the composition according to the invention (Example 1) was tested on date palm seedlings obtained from the germination of the stones of dates of the Medjool variety. Seedlings in leaf stage received different doses of the composition of Example 1 according to the invention (0; 1.65; 3.33; 6.67 g/l). After 4 months of treatment (at a rate of twice per week), the seedlings were harvested, and the wet and dry weights of the root portion of each seedling were determined.

The dilutions used are:

1.65 g of the composition in 1 l of water

3.3 g of the composition in 1 l of water

6.67 g of the composition in 1 l of water

The application mode is by irrigation.

The results are presented in Table 8.

TABLE 8
Stimulating Effect of the Rhizogenesis of the Composition
According to the Invention in Date Palm Seedlings

| | Wet Weight in g | Dry Weight in g |
|---|---|---|
| PNT | 3.9 ± 0.4 | 1.1 ± 0.1 |
| PT 1.65 g/l | 5.0 ± 0.8 | 1.8 ± 0.2 |
| PT 3.3 g/l | 4.4 ± 0.4 | 1.3 ± 0.2 |
| PT 6.67 g/l | — | — |

These results reveal the stimulating effect of the rhizogenesis of the composition according to the invention in date palm seedlings; the two doses 1.65 and 3.33 g/l have proven the most effective, with the dose 1.65 g/l being superior compared to the control seedlings that have received only water throughout the experiment. Further, the dose 6.67 g/l has proven phytotoxic in the cells of the root system of the young seedlings.

### Effect of the Composition According to the Invention on the Post-Harvest Preservation of Dates

So as to verify the feasibility of the use of the composition according to the invention on the post-harvest preservation of dates, testing was carried out. This testing consisted in soaking the dates for 10 minutes in solutions containing various concentrations of the composition of Example 1 (0; 3.33, and 6.6 g/l). Next, the dates were rinsed with water, and then dried before being stored in hermetically-sealed plastic boxes. For the purpose of evaluating the bacterial load (FMAT) and the fungal load of the treated dates, sampling by swabbing eight dates from each box was carried out. The sampling surface is 1 cm² per date.

The dilutions used are:

3.33 g of the composition in 1 l of water

6.6 g of the composition in 1 l of water

The application mode is by soaking.

The results obtained are presented in Tables 9 and 10.

TABLE 9
Bacterial Load Contained in the Dates Treated with the Composition
According to the Invention and Those Not Treated (ufc/cm²)

| | Untreated | Treated Dates | Treated Dates |
|---|---|---|---|
| | Dates | 3.3 g/l | 6.6 g/l |
| First Day | 2.1 ± 0.9 | 0.0 ± 0.0 | 0.0 ± 0.0 |
| After One Month | 12.6 ± 1.3 | 1.8 ± 1.1 | 0.0 ± 0.0 |
| After Three Months | Tp | 17.8 ± 2.6 | 4.6 ± 1.6 |
| After Six Months | Tp | Tp | 11.7 ± 1.2 |
| After Nine Months | Tp | Tp | Tp |

Tp: Uncountable Layers

TABLE 10
Fungal Load Contained in the Dates Treated with the Composition
According to the Invention and Those Not Treated (ufc/cm²)

| | Untreated | Treated Dates | Treated Dates |
|---|---|---|---|
| | Dates | 3.3 g/l | 6.6 g/l |
| First Day | 7 ± 1.75 | 1 ± 0.5 | 0.0 ± 0.0 |
| After One Month | 18.5 ± 3.0 | 2.6 ± 0.8 | 0.0 ± 0.0 |
| After Three Months | Tp | 24 ± 2.7 | 8.2 ± 1.5 |
| After Six Months | Tp | Tp | 20.3 ± 2.1 |
| After Nine Months | Tp | Tp | Tp |

Tp: Uncountable Layers

Tables 9 and 10 respectively present the results of the bacterial load and the fungal load contained in the treated dates and those that are not treated. The dose 6.67 g/1l has proven the most effective since the soaked dates at this dose are free of bacteria and fungi that can interfere with their preservation for a month and at ambient temperature.

### Effect of the Composition According to the Invention on the Vase Life of Cut Flowers

The composition according to the invention (Example 1) was also tested on the vase life of cut flowers and more specifically of carnations. To do this, the carnation flowers were cut to equal lengths of 55 cm before being put to soak in solutions containing two concentrations of the composition according to the invention (1.6 and 3.3 g/l) immediately after harvest.

The measurements taken into consideration for evaluating the effect of the composition according to the invention are:
Weight of the flower stem every 4 days
Percentage of opening of the flower buds

The dilutions used are:

1.6 g of the composition in 1 l of water

3.3 g of the composition in 1 l of water

The application mode is as follows: putting into a vase.

The results obtained are presented in Tables 11 and 12.

TABLE 11
Effect of the Composition According to the Invention on the Weight of Flower Stems
in a Vase

| | First Day | Fourth Day | Eighth Day | 12^{th} Day | 16^{th} Day |
|---|---|---|---|---|---|
| Water | 27.46 ± 1.4 g | 31.36 ± 1.2 g | 31.65 ± 1.36 g | 30.16 ± 1.12 g | 28.7 ± 1.42 g |
| Composition | 28.26 ± 1.11 g | 30.93 ± 0.75 g | 31.35 ± 0.98 g | 31.93 ± 1.09 g | 32.56 ± 0.86 g |
| 1.6 g/l | | | | | |
| Composition | 29.28 ± 1.02 g | 32.45 ± 0.65 g | 32.71 ± 0.92 g | 34.11 ± 0.76 g | 34.52 ± 1.21 g |
| 3.3 g/l | | | | | |

TABLE 12
Effect of the Composition According to the Invention on the
Opening of Flower Buds

| | First Day | | | Eighth Day | | | 16^{th} Day | | |
|---|---|---|---|---|---|---|---|---|---|
| | | | % of | | | % of | | | % of |
| | F | O | Opening | F | O | Opening | F | O | Opening |
| Water | 24 | 0 | 0 | 19 | 5 | 21 | 18 | 6 | 25 |
| Com- | 21 | 0 | 0 | 13 | 8 | 38 | 12 | 9 | 43 |
| position | | | | | | | | | |
| 1.6 g/l | | | | | | | | | |
| Com- | 25 | 0 | 0 | 18 | 7 | 28 | 15 | 10 | 40 |
| position | | | | | | | | | |
| 3.3 g/l | | | | | | | | | |

These results reveal that the composition according to the invention improves the vase life of the cut flowers.

### In Vitro Test of the Antimicrobial Activity of the Composition According to the Invention

The effectiveness of the composition according to the invention (Example 1) was tested on the microbial load (bacteria, yeasts and molds, parasites) of the water used in watering poultry. The sample is moved to the laboratory and preserved wet for the in vitro test.

The culture media used are:
PCA (Plate Count Agar) (Biokar) for the cultivation and testing of the antibacterial activity of the revivable aerobic bacteria.
Desoxycholate lactose agar (Biokar) for the cultivation and testing of the antibacterial activity of total and fecal coliforms.
Slanetz agar for the cultivation and testing of the antibacterial activity of staphylococci.
TSC (Tryptose Sulfite Cycloserine Agar) (Biokar) for the cultivation and testing of the antibacterial activity of anaerobic bacteria.
Wilson-Blair agar (Biokar) for the cultivation and testing of the antibacterial activity of salmonella.
Sabouraud Chloramphenicol agar (Biokar) for the cultivation and testing of the antifungal activity of yeasts and molds.

The testing of the antibacterial activity of the composition according to the invention was carried out as follows:
Various concentrations of the composition of Example 1 (1 g/l; 2 g/l; 4 g/l) were added to the water sample. A negative control containing just the water sample is also prepared.
The culture media were prepared according to the instructions of the supplier, sterilized in the autoclave for 15 minutes at 121° C. and cooled to 45° C. They are then poured into Petri dishes (90×16 mm) and allowed to cool to 4° C. for 24 hours, and then inoculated with a volume of 100 μl of the water sample. The dishes are incubated at 37° C. for 24 hours for the revivable aerobic bacteria, total coliforms, salmonella, staphylococci, and anaerobic bacteria. For the fecal coliforms, the dishes are incubated at 44° C. for 24 hours.

The testing of the antifungal activity of the composition according to the invention was carried out by following the same protocol as that produced in the testing of the antibacterial activity except that the incubation temperature is on the order of 27° C., and the incubation time is from 3 to 5 days.

Regarding the testing of the antiparasitic activity, a volume of 10 μl of a water sample was placed between a thin glass strip and the Malassez cell. The number of cells in 10 rectangles was then counted.

The results obtained are presented in Table 13.

TABLE 13
Effect of the Composition According to the Invention on the Reduction of the
Microbial Load of Surface Water

| | Control | 1 g/l | 2 g/l | 4 g/l |
|---|---|---|---|---|
| Revivable | 2.67 ± 0.17 10⁵ | 1.8 ± 0.37 10⁴ | 3.93 ± 0.51 10³ | 3 ±1 |
| Aerobes | | | | |
| (UFC/ml) | | | | |
| Total Coliforms | 1.15 ± 0.1 10⁴ | 1.09 ± 0.8 10⁴ | 2.05 ± 0.43 10³ | 0 |
| (UFC/ml) | | | | |
| Fecal Coliforms | 1.51 ± 0.2 10³ | 5.26 ± 1.92 10² | 43.33 ± 15.27 | 1.33 ± 1.52 |
| (UFC/ml) | | | | |
| Salmonella | 1.13 ± 0.35 10⁸ | 1.81 ± 0.21 10⁵ | 1.2 ± 0.4 10⁴ | 0 |
| (UFC/ml) | | | | |
| Staphylococci | 6.06 ± 1.22 10² | 5.86 ± 0.7 10² | 40 ± 8 | 3.66 ± 1.52 |
| (UFC/ml) | | | | |
| Anaerobic | 81 ± 14.93 | 17.66 ± 8.62 | 2.33 ± 0.57 | 0 |
| Bacteria | | | | |
| (UFC/ml) | | | | |
| Yeasts and | 3.06 ± 0.59 10³ | 1.33 ± 0.28 10² | 56.66 ± 11.5 | 0 |
| Molds (UFC/ml) | | | | |
| Parasites | 1.33 ± 0.17 10⁴ | 1.79 ± 0.18 10³ | 1.16 ± 0.35 10³ | 5.23 ± 2.01 10² |
| (cells/ml) | | | | |

These results reveal that the treatment of the surface water by the composition brings about a significant reduction in the microbial load. This reduction increases with the concentration of the composition.

### In Vivo Testing of the Antimicrobial Activity of the Composition According to the Invention

The composition according to the invention was also tested in vivo on chicks. Twelve chicks per batch were used in this testing. During the entire period of the testing, the temperature of the animal house was adjusted to 28° C., and the animals had free access to water and to the feed that was continuously available.

The water used in this testing is the surface water sampled from the reservoir for distribution of watering water that was used in the in vitro testing.

The objectives of this testing are:
To test in vivo the effect of the treatment of watering water by the composition of Example 1 on the behavior of animals
To compare in vivo the effect of the composition of Example 1 on non-stagnant fresh water and on stagnant water

The distribution of the batches is as follows:

| | | | Initial |
|---|---|---|---|
| | | | Number of |
| | Feed | Watering | Chicks |
| ENS Control | Blank Feed | Untreated, Non- | 12 |
| | | Stagnant Water | |
| ENS 1 g/l | Blank Feed | Treated, Non- | 12 |
| | | Stagnant Water 1 g/l | |
| ENS 2 g/l | Blank Feed | Treated, Non- | 12 |
| | | Stagnant Water 2 g/l | |
| ES Control | Blank Feed | Untreated, Stagnant | 12 |
| | | Water | |
| ES 1 g/l | Blank Feed | Treated, Stagnant | 12 |
| | | Water 1 g/l | |
| ES 2 g/l | Blank Feed | Treated, Stagnant | 12 |
| | | Water 2 g/l | |

ENS: Non-Stagnant Water
ES: Stagnant Water

To evaluate the effectiveness of the composition, the measurements of multiple zootechnical types during the following three breeding phases were compared:
The mean live weight: each batch of chicks is weighed from the first day. Next, individual weigh-ins are carried out at the end of each phase. The mean live weight is determined as follows:

Mean live weight (g)=weight of all of the chicks of one batch/number of chicks of this batch
The increase in live weight: the chicks from each batch were weighed upon arrival to calculate the mean weight upon start-up. Later, the weigh-ins were carried out at the end of each breeding phase up to the end of the experiment. The increase in weight is determined by the following formula:

Increase in Live Weight (g)=P2−P1
P2: final mean weight of a breeding phase
P1: initial mean weight of this breeding phase
The food consumption: the various feeds are weighed and distributed daily up to the 35^{th} day. The rest of the feed contained in the troughs is weighed daily for each batch. Thus, the quantities of feed consumed per batch were recorded at the end of each breeding phase. The mean quantity of feed consumed was determined by the following formula:

$$Food consumption ( g ) = \frac{\begin{matrix}Quantity of feed distributed - \\ Quantity of remaining feed\end{matrix}}{Number of chicks}$$

The consumption index (IC): corresponds to the quantity of feed necessary for producing 1 kilogram of live weight. The IC is determined for each phase by the following formula:

$$IC = \frac{Quantity of feed consumed during one phase}{Increase in mean weight of this phase}$$

The mortality rate: the mortality is noted daily. The mortality rate is determined for each batch during a given phase by the following formula:

$$Mortality rate ( \% ) = \frac{\begin{matrix}Numbers of dead chicks \\ during a phase \times 100\end{matrix}}{\begin{matrix}Total numbers of chicks \\ during this phase\end{matrix}}$$

The results obtained are presented in Table 14.

TABLE 14
Effect of the Composition According to the Invention on the Mean Live Weight and
the Increase in Mean Weight

| | Mean Live Weight (g) | | | | Increase in Mean Weight (g) | | |
|---|---|---|---|---|---|---|---|
| | | Start-Up | Increase | Finish | Start-Up | Increase | Finish |
| | D0 | (D1-D7) | (D7-D21) | (D22-35) | (D1-D7) | (D7-D21) | (D22-35) |
| ENS | 37.16 ± 2.08 | 70.83 ± 8.83 | 214 ± 8.43 | 444 ± 23.66 | 33.67 | 143.17 | 230 |
| Control | | | | | | | |
| ENS | 36.58 ± 3.84 | 80.41 ± 7.86 | 246.36 ± 32.33 | 680 ± 41.47 | 43.83 | 165.95 | 433.64 |
| 1 g/l | | | | | | | |
| ENS | 36.83 ± 3.78 | 86.63 ± 16.6 | 317 ± 9.48 | 725 ± 27.98 | 49.8 | 230.37 | 408 |
| 2 g/l | | | | | | | |
| ES | 36.41 ± 2.67 | 65.2 ± 10.4 | 168 ± 40.24 | 366 ± 60.66 | 28.79 | 102.8 | 198 |
| Control | | | | | | | |
| ES | 36.66 ± 2.8 | 84.75 ± 12.33 | 214.28 ± 7.86 | 560 ± 90.11 | 48.09 | 129.53 | 345.72 |
| 1 g/l | | | | | | | |
| ES | 37.75 ± 2.95 | 75.09 ± 13 | 242 ± 24.85 | 631 ± 18.52 | 37.34 | 166.91 | 389 |
| 2 g/l | | | | | | | |

ENS: Non-Stagnant Water
ES: Stagnant Water

TABLE 15
Effect of the Composition According to the Invention on the Food Consumption,
Consumption Index, and Mortality Rate

| | Food Consumption (g) | | | Consumption Index | | | Mortality Rate (%) | | |
|---|---|---|---|---|---|---|---|---|---|
| | Start-Up | Increase | Finish | Start-Up | Increase | Finish | Start-Up | Increase | Finish |
| | (D0-D7) | (D8-D21) | (D22-D40) | (D0-D7) | (D8-D21) | (D22-D40) | (D0-D7) | (D8-D21) | (D22-D40) |
| ENS | 75 | 270 | 914 | 2.23 | 1.89 | 3.97 | 0/12 | 2/12 | 1/12 |
| Control | | | | | | | (0%) | (16.6%) | (8.3%) |
| ENS | 61.66 | 245.45 | 931.8 | 1.41 | 1.48 | 2.14 | 0/12 | 1/12 | 0/12 |
| 1 g/l | | | | | | | (0%) | (8.3%) | (0%) |
| ENS | 69.09 | 270 | 990 | 1.39 | 1.17 | 2.42 | 1/12 | 0/12 | 0/12 |
| 2 g/l | | | | | | | (8.3%) | (0%) | (0%) |
| ES | 208 | 260 | 950 | 7.22 | 2.53 | 4.79 | 7/12 | 0/12 | 0/12 |
| Control | | | | | | | (58.33%) | (0%) | (0%) |
| ES | 81.25 | 375.71 | 1,275 | 1.69 | 2.90 | 3.68 | 4/12 | 5/12 | 0/12 |
| 1 g/l | | | | | | | (33.33%) | (41.6%) | (0%) |
| ES | 79.09 | 270 | 1,030 | 2.12 | 1.62 | 2.64 | 1/12 | 1/12 | 0/12 |
| 2 g/l) | | | | | | | (8.3%) | (8.3%) | (0%) |

ENS: Non-Stagnant Water
ES: Stagnant Water

These results reveal that the animals treated by the composition according to the invention do better than the untreated animals; the dose that provided the best results in terms of zootechnical types is 2 g/1.

### Effect of the Composition According to the Invention on the Reduction of the Intestinal Load of the Animals (Broiler Chicks)

The effect of the composition according to the invention (Example 1) was tested on the reduction of the intestinal load (revivable aerobic bacteria and the parasite load) of the broiler chicks for 35 days. Every 7 days, the samples of droppings from various batches were sampled and solubilized in physiological water (1 g of droppings in 9 ml of physiological water).

Dilutions are then prepared from the stock solution.

The culture medium that is used is:
PCA (Plate Count Agar) (Biokar) for the cultivation of revivable aerobic bacteria.
The culture medium was prepared according to the instructions of the supplier, sterilized in the autoclave for 15 minutes at 121° C. and cooled to 45° C., and then poured into Petri dishes (90×16 mm) and allowed to cool to 4° C. for 24 hours, and then inoculated with a volume of 100 l of the sample. The dishes are incubated at 37° C. for 24 hours. The test is performed in triplicate.

Regarding the parasite load, a volume of 10 μl of the sample is placed between a thin glass strip and the Malassez cell. The number of cells in 10 rectangles is then counted. The results are presented in Tables 16 and 17.

TABLE 16
Effect of the Composition According to the Invention on the Reduction of the
Intestinal Load of Revivable Aerobic Bacteria

| | ENS Control | ENS 1 g/l | ENS 2 g/l | ES Control | ES 1 g/l | ES 2 g/l |
|---|---|---|---|---|---|---|
| D1 | 23 ± 2.8 10⁹ | | | | | |
| D7 | 11 ± 0.5 10¹⁰ | 25 ± 5 10⁹ | 13 ± 2.8 10⁹ | 15.6 ± 1.04 10¹⁰ | 44.5 ± 35.8 10⁹ | 38.3 ± 18.9 10⁹ |
| D14 | 13.5 ± 2.7 10¹⁰ | 23.3 ± 12.5 10⁹ | 8.3 ± 10.4 10⁹ | 11.6 ± 2.8 10¹¹ | 6.1 ± 0.2 10¹⁰ | 26.6 ± 15.2 10⁹ |
| D21 | 7 ± 1.5 10¹⁰ | 6.6 ± 5.7 10⁹ | 5 ± 5 10⁹ | 11 ± 1 10¹⁰ | 13.3 ± 12.5 10⁹ | 11.6 ± 2.8 10⁹ |
| D28 | 8.5 ± 2.6 10¹⁰ | 2 ± 1.3 10¹⁰ | 8.3 ± 5.7 10⁹ | 16.1 ± 3.6 10¹⁰ | 9 ± 2.5 10¹⁰ | 15 ± 10 10⁹ |
| D35 | 11.6 ± 3 10¹⁰ | 25 ± 5 10⁹ | 11.6 ± 2.8 10⁹ | 14.1 ± 2.8 10¹⁰ | 6.8 ± 1.8 10¹⁰ | 25 ± 10 10⁹ |

TABLE 17
Effect of the Composition According to the Invention on the Reduction of the
Intestinal Parasite Load

| | ENS Control | ENS 1 g/l | ENS 2 g/l | ES Control | ES 1 g/l | ES 2 g/l |
|---|---|---|---|---|---|---|
| D1 | 41.5 ± 9.1 10¹¹ | | | | | |
| D7 | 2.2 ± 2.2 10¹³ | 1.4 ± 0.8 10¹² | 1.7 ± 0.6 10¹¹ | 1.6 ± 1.9 10¹³ | 2.1 ± 0.8 10¹³ | 1.7 ± 0.1 10¹¹ |
| D14 | 9.5 ± 4.9 10¹² | 7.9 ± 4.1 10¹² | 2.1 ± 0.2 10¹⁰ | 1 ± 0.4 10¹³ | 6.1 ± 0.5 10¹² | 7.5 ± 0.4 10¹¹ |
| D21 | 1.3 ± 0.5 10¹³ | 5.5 ± 0.9 10¹² | 3 ± 1.5 10¹⁰ | 2.3 ± 0.3 10¹³ | 1.7 ± 0.2 10¹³ | 4.3 ± 4.6 10¹¹ |
| D28 | 1.23 ± 0.2 10¹³ | 6.05 ± 3.3 10¹² | 1.5 ± 0.5 10⁹ | 1.6 ± 0.2 10¹³ | 9.5 ± 1.6 10¹² | 7.2 ± 3.6 10¹⁰ |
| D35 | 10.2 ± 0.4 10¹² | 6.7 ± 1.1 10¹² | 1.3 ± 0.7 10¹⁰ | 1.9 ± 0.2 10¹³ | 15 ± 0.5 10¹² | 69 ± 0.7 10¹¹ |

The results that are obtained clearly reveal that the animals that are treated by the composition according to the invention have a lower intestinal load in relation to untreated animals; the dose that provided the best results in terms of the reduction of the intestinal load is 2 g/l.

Comparison Study Between the Antibacterial Action of the Composition According to the Invention and Glutaraldehyde Against E. coli In Vitro.

In microplates with 96 wells each containing 130 μl of sterile Muiller-Hinton stock (autoclaving for 15 minutes at 110° C.), a variable volume of the stock solution of glutaraldehyde [1/10 (100 mg/ml), 1/100 (10 mg/ml), 1/250 (4 mg/ml), 1/500 (2 mg/ml), 1/1,000 (1 mg/ml), 1/2,000 (0.5 mg/ml), 1/4,000 (0.25 mg/ml), 1/8,000 (0.125 mg/ml), and 1/16,000 (0.062 mg/ml)] and of the composition according to the invention of Example 1 [1/10 (133.4 mg/ml), 1/100 (66.7 mg/ml), 1/250 (26.68 mg/ml), 1/500 (13.34 mg/ml), 1/1,000 (6.67 mg/ml), 1/2,000 (3.33 mg/ml), 1/4,000 (1.66 mg/ml), 1/8,000 (0.83 mg/ml), and 1/16,000 (0.42 mg/ml)] was added so as to obtain the final concentrations for each disinfectant. After, 20 μl of the inoculum was added into each well. Positive and negative controls were also prepared. The plates are then incubated at 37° C. for 24 hours. The bacterial growth was tracked by the optical density using the spectrophotometer with a 600-nm wavelength.

The results are presented in Table 18.

TABLE 18
Comparison Between the Action of Glutaraldehyde and the
Composition According to the Invention Against E. coli

| | | Composition According to |
|---|---|---|
| Concentrations | Glutaraldehyde | the Invention |
| 1 | 0 | 0 |
| 1/10 | 0.13 ± 0.002 | 0 |
| 1/100 | 0.15 ± 0.002 | 0 |
| 1/250 | 0.17 ± 0.003 | 0.02 ± 0.0007 |
| 1/500 | 0.20 ± 0.002 | 0.09 ± 0.007 |
| 1/750 | 0.20 ± 0.002 | 0.11 ± 0.010 |
| 1/1,000 | 0.22 ± 0.012 | 0.11 ± 0.006 |
| 1/2,000 | 0.28 ± 0.035 | 0.25 ± 0.016 |
| 1/4,000 | 0.34 ± 0.007 | 0.31 ± 0.008 |
| 1/8,000 | 0.47 ± 0.034 | 0.45 ± 0.017 |
| 1/16,000 | 0.50 ± 0.005 | 0.49 ± 0.008 |
| Positive Control | 0.52 ± 0.017 | 0.52 ± 0.008 |
| Negative Control | 0 | 0 |

These results present the effect of glutaraldehyde and the preparation of the HE at various concentrations on an E. coli suspension on the order of 10⁸ UFC/ml. They reveal that the composition according to the invention is more effective than the disinfectant (glutaraldehyde). The inhibiting minimal concentration (CMI) is 1/100 for the composition according to the invention, whereas for the glutaraldehyde, the CMI is obtained only with the pure disinfectant. A more significant partial inhibition is noted for the 1/250 to 1/1,000 concentrations of the composition according to the invention in relation to glutaraldehyde.

### Antibacterial Activity of Glutaraldehyde and of the Composition According to the Invention in the Disinfection of the Fibroscope.

So as to compare the antibacterial action of the composition according to the invention and of glutaraldehyde, the following steps were carried out:
Artificial contamination: 6 ml of the E. coli suspension that was previously prepared is introduced using a syringe into the proximal opening of the operating channel, and into the proximal opening of the intake channel (Dusseau and coll., 2001). Packaging of the fibroscope and incubation for 20 minutes at 37° C.
Intermediate rinsing with distilled water.
First sampling (before disinfection).
First cleaning.
Rinsing by tap water for 2 minutes.
Second cleaning.
Disinfection either by glutaraldehyde or by the composition according to the invention.
Final rinsing.
Second sampling (after disinfection) carried out under the same conditions and following the same steps as during the first sampling.

The results obtained are presented in Table 19.

TABLE 19
Antibacterial Action Between the Two Products that are Tested.

| | | Composition | Composition |
|---|---|---|---|
| Samplings | Glutaraldehyde | According to the | According to the |
| Disinfectants | (1/10) | Invention (1/1,000) | Invention (1/500) |
| Before | Layers | Layers | Layers |
| Disinfection | | | |
| After | Layers | 50 Colonies | 0 Colonies |
| Disinfection | | | |

The results obtained revealed the presence of a layer of bacteria before disinfection; the same result was obtained after the disinfection by glutaraldehyde, whereas after the disinfection by the composition according to the invention, the bacterial load was only 50 UFC/ml with the 1/1,000 concentration and zero with the 1/500 concentration.

Further, these results reveal a significant reduction of the microbial load of the fibroscope after the disinfection by the composition according to the invention. This shows the advantage of using the composition according to the invention as an alternative treatment to glutaraldehyde.

### Effect of the Composition According to the Invention on Post-Harvest Fruits

Drenching and Waxing Treatment of Clementines (“Afourar” Variety). Fruits Experimentally Infected by Injection of Spores
Six batches of 10 fruits of the “Afourar” variety were treated with various preparations:
Batch 1: Contains 10 fruits, washed with a paste consisting of chemical fungicides (imazalil at a dose of 500 cc/hl and orthophenylphenol at a dose of 750 ppm).
Batch 2: Contains 10 fruits, washed with a paste consisting of the composition according to the invention at a dose of 3 kg per ton of water.
Batch 3: Contains 10 fruits, waxed with a mixture of wax and imazalil at a dose of 3,000 ppm, and then dried in hot air (50° C.).
Batch 4: Contains 10 fruits, waxed with a mixture of wax and the composition according to the invention at a dose of 10 kg per ton, and then dried (50° C.).
Batch 5: Contains 10 fruits, washed with a paste consisting of chemical fungicides (imazalil at a dose of 500 cc/hl and orthophenylphenol at a dose of 750 ppm); after their drying, the latter were waxed with a mixture of wax and imazalil at a dose of 3,000 ppm, and then dried (50° C.).
Batch 6: Contains 10 fruits, washed with a paste consisting of the composition according to the invention at a dose of 3 kg per ton of water; after their drying, the latter were waxed with a mixture of wax and the composition according to the invention at a dose of 10 kg per ton, and then dried (50° C.).
   Washing was done using a shower head that makes it possible to wash the fruits in a way that is comparable to that of the drench chamber in the packing station.
   Waxing was done by spraying in a way that is comparable to that of the waxing system in the packing station.

24 hours after the treatment, each piece of fruit of the six batches was infected by injection of a volume of 100 μl of a suspension of spores of the Penicillium digitatum strain (10⁶ spores/ml), using a syringe equipped with a very fine needle (30-gauge), inclined tangential to the albedo surface. The injection point is marked by a circle traced with an indelible marker. The fruits were then incubated at a temperature of 20° C.

Daily monitoring of the six batches was carried out, and the appearance as well as the development of rot at the injection points was noted for the fruits of each batch.

The results obtained reveal that rot begins starting from the third day after infection in the batch washed with the chemical fungicides, whereas in the batch washed with the composition according to the invention, rot begins only after four days. The results also reveal that the rot in the batch washed with the chemical fungicides develops in a faster way with a diameter of 3.2 cm±0.7 after 7 days, in relation to the batch washed with the composition according to the invention where the diameter of the rot reaches only 2 cm±0.6 after 7 days. The results also reveal a delay in the appearance of spores in the batch treated with the composition according to the invention in comparison with the batch treated with chemical fungicides.

For the batches 3 and 4, rot begins starting from the fourth day after infection in the batch treated with the mixture of the wax plus the composition of the invention and the batch treated with the mixture of the wax plus imazalil. However, a faster development of rot was noted in the batch treated with the mixture of wax and imazalil (2.55 cm±0.3 in diameter after 7 days), in relation to the development of rot in the batch treated with the mixture of wax and the composition according to the invention (1.66 cm±0.3 in diameter after 7 days).

For the batches 5 and 6: Rot in the batch that is washed and waxed with the preparation of the invention (1.2 cm±0.4 in diameter after 7 days) develops sparingly in relation to the batch that is washed and waxed with chemical fungicides (2.07 cm±0.5 in diameter after 7 days). Similar results were obtained for the “Maroc Late” variety.

Therefore, the preparation of the invention makes possible a better protection than the chemical fungicides when it is used for washing and waxing fruits that are experimentally infected with an extreme number of spores located in the injection point.

Drenching and Waxing Treatment of Clementines (“Afourar” Variety). Non-Infected, Experimentally Damaged Fruits

Three batches of 10 fruits of the “Afourar” variety were prepared:
Batch 1: 10 fruits washed with a paste consisting of chemical fungicides (imazalil at a dose of 500 cc/hl and orthophenylphenol at a dose of 750 cc/hl), next waxed with a mixture of wax plus imazalil at a dose of 3,000 ppm, and then dried at 50° C.
Batch 2: 10 fruits washed with a paste consisting of the composition according to the invention at a dose of 3 kg per ton of water, next waxed with a mixture of wax plus the preparation of the invention at a dose of 10 kg per ton, and then dried at 50° C.
Batch 3: 10 fruits washed with just water, next waxed with just wax, and then dried at 50° C.

Two equidistant holes (diameter 1 mm+depth 2 mm) that pass through the flavedo and the albedo and that extend to the pulp were made on each piece of fruit. Next, these fruits were preserved in hermetically-sealed boxes and saturated with moisture. The boxes were incubated at a temperature of 27° C.

The percentage of the surface of rotted fruits at the holes was noted for the fruits of each batch.

The results are presented in Table 20.

TABLE 20
Percentage of the Rotten Surface on the Fruits Treated by the Composition of the
Invention in Relation to the Fruits Treated with Imazalil and the Untreated Fruits:

| | Days | | | | | | |
|---|---|---|---|---|---|---|---|
| Batches | 4 D | 6 D | 8 D | 10 D | 12 D | 14 D | 16 D |
| Batch 1: Fruits washed with tap water | 12% | 36% | 50% | 61.13% | 73% | 90% | 100% |
| and then waxed with just wax | | | | | | | |
| Batch 2: Fruits washed with water and | 0% | 7% | 11% | 17% | 29% | 38% | 44% |
| imazalil and then waxed with the mixture | | | | | | | |
| of the wax plus imazalil | | | | | | | |
| Batch 3: Fruits washed with water and | 0% | 0% | 0% | 0% | 2% | 2% | 6% |
| the composition according to the | | | | | | | |
| invention and then waxed with the | | | | | | | |
| mixture of the wax and of the | | | | | | | |
| composition according to the invention | | | | | | | |

The results reveal a very significant reduction in the percentage of rotten surface in fruits treated with the preparation of the invention in relation to the control fruits treated with water and just wax. This reduction is also noteworthy in relation to the fruits treated with chemical fungicides.

These results reveal that the composition according to the invention makes possible a preservation of the fruits for 12 days without rot whereas for the batch treated with chemical fungicides, rot appeared on the 6^{th} day. For the batch without treatment, rot appeared on the 4^{th} day.

Treatment by Soaking Clementines, Directly on Site at a Citrus Fruit Farm

Field tests were carried out at a citrus fruit farm. In these tests, were used a batch of four boxes of fruits of the “Afourar” variety that were soaked immediately after being picked up in the water containing the preparation of the invention at the dose of 1 g per ton, another batch of 4 boxes of fruit soaked immediately after being picked up in just water. 30 fruits were sampled from each batch, and the fungal load of each piece of fruit was evaluated by the number of spores (units forming a colony) per square centimeter of the surface of the fruit.

The results obtained reveal that the fungal load of the fruits soaked in a solution with the composition according to the invention is on the order of 10⁵ spores per cm², smaller than the fungal load of the fruits soaked in the water itself that reaches 10⁸ spores per cm². These results reveal that the fruits soaked in the preparation of the invention will arrive at the packing station with a more reduced initial load (1,000 times smaller) in relation to that of the fruits soaked in just water.

Drenching and Waxing on Infected Clementines, Tests in a Packing Station

Field tests were carried out in a station for packing citrus fruit. In these tests, a batch of 6 tons of fruits of the “Afourar” variety was washed in the drench chamber with a paste that contains water and the preparation of the invention at a dose of 3 kg per ton; next, in the waxing step, this same batch was waxed with a mixture of wax and the preparation of the invention at a dose of 5 kg per ton. Another batch of 6 tons of fruit of the “Afourar” variety was washed in the drenching step with a paste consisting of chemical fungicide (imazalil at a dose of 500 cc/hl and orthophenylphenol at a dose of 750 c/hl), and next waxed with the mixture of the wax and the imazalil at a dose of 3,000 ppm. 30 fruits from each batch were sampled after the drenching step and 30 fruits after the waxing step so as to evaluate their fungal load.

The results are presented in Table 21.

TABLE 21
Fungal Load of Fruits Treated with the Composition According
to the Invention or with the Chemical Fungicides in the
Drenching Step and in the Waxing Step. Load Expressed in
Terms of Number of Spores (UFC/cm2) per cm2

| | Drenching | Waxing |
|---|---|---|
| Fruit Treated with the | 5 10³ ± 1.2 | 30 ± 4 |
| Composition According to | | |
| the Invention | | |
| Fruit Treated with Imazalil | 7 10⁶ ± 2.5 | 6 10³ ± 0.96 |

The results obtained reveal that there is a significant difference in the fungal load of fruits treated with the composition according to the invention, which is 1,000 times smaller than that of the fruits treated with the chemical fungicides after the drenching step and after the waxing step.

Tracking of the Rot Rate of Clementines in a Packing Station

Field tests were performed in a citrus fruit packing station. In these tests, a first batch of 6 tons (imazalil batch) of fruits of the “Afourar” variety was washed with a paste consisting of chemical fungicides (imazalil at a dose of 500 cc/hl and orthophenylphenol at a dose of 750 cc/hl) in the drenching step, and waxed with a mixture of the wax and imazalil at a dose of 300 ppm in the waxing step. A second batch of 6 tons of fruits of the “Afourar” variety (composition batch) was washed with a paste containing water and the composition according to the invention at the dose of 3 kg per ton, and waxed with a mixture of wax and the composition according to the invention at the dose of 5 kg per ton. After being put into boxes, two sub-batches of 15 boxes were sampled from each batch and distributed as follows:
Sub-batch a: 15 boxes of the imazalil batch, stored at 8° C.
Sub-batch b: 15 boxes of the composition batch stored at 8° C.
Sub-batch c: 15 boxes of the imazalil batch stored at 25° C.
Sub-batch d: 15 boxes of the composition batch stored at 25° C.

The sub-batches were monitored once per week for a month. A count of rotten fruits in each batch makes it possible to detect the change in the rate of rotting depending on the storage temperature and the treatment used.

The results are presented in Table 22.

TABLE 22
Percentage of Rotten Fruits Depending on the Treatment
Used Under Two Different Storage Conditions

| | Week 1 | Week 2 | Week 3 | Week 4 |
|---|---|---|---|---|
| Sub-Batch a | 2.63% | 1.35% | 2.16% | 4.67% |
| Storage at 8° C. | | | | |
| Sub-Batch b | 0.56% | 0.50% | 1.01% | 2.82% |
| Storage at 8° C. | | | | |
| Sub-Batch c | 7.78% | 6.91% | 9.68% | 8.43% |
| Storage at 25° C. | | | | |
| Sub-Batch d | 4.50% | 4.65% | 5.63% | 5.63% |
| Storage at 25° C. | | | | |

The results obtained reveal that the rate of rotting in the sub-batches preserved at a temperature of 25° C. is higher in comparison with the sub-batches preserved at a temperature of 4° C. The results also reveal that the fruits treated with the composition according to the invention have a rate of rotting of between 0.5% and 2.82% at a temperature of 4° C., and between 4.5% and 6.91% at 25° C.; this rate is lower in relation to the rate of rotting in fruits treated with the chemical fungicides, which varies between 1.35% and 4.67% at a storage temperature of 4° C., and between 4.62% and 9.68% at a temperature of 25° C.

Treatment of Ambient Air in a Clementine Packing Station

Tests were carried out in a citrus fruit packing station. In these tests, the ambient air from various zones of the station was treated by spraying the preparation of the invention at a dose of 10 kg per ton of water twice per day; between 12 hours and 13 hours and at 18 hours. Air samples (Petri dish with open PDA medium for 5 minutes) were taken so as to evaluate the fungal load of the ambient air of each zone of the station before and after treatment and this four times per day:
At 0730 hours, before the beginning of the activity of the workers; 13 hours after the treatment of the previous day,
At 1200 hours, before the first treatment of the air (1230 hours) during the workers' break,
At 1330 hours, one hour after the first treatment,
At 1800 hours, before the 2^{nd} treatment (1830 hours).

The results obtained are presented in Table 23.

TABLE 23
Number of Colonies per Petri Dish

| | 0700 Hours | 1200 Hours | 1300 Hours | 1800 Hours |
|---|---|---|---|---|
| Air from the | 06 | 32 | 04 | 08 |
| Packaging Zone | | | | |
| Air from the | 10 | 20 | 09 | 16 |
| Sorting Zone | | | | |
| Air from the | 0 | 07 | 05 | 17 |
| Storage Area | | | | |

The results reveal that the fungal load of the air from the various zones was very low at the beginning of the day due to the treatment of the air with the preparation of the invention carried out at the end of the preceding day. During the day, this load gradually increases up until noon or a spraying of the preparation of the invention causes a significant reduction of this load. From the afternoon until the end of the day, a slight increase in the fungal load of the ambient air from various zones was noted which will be decreased by a second spraying of the preparation of the invention. This approach makes it possible to pack the fruits without the risk of contamination by the microflora of the air.

Evaluation of the Effect of the Composition According to the Invention in the Laboratory

A strain of penicillium digitatum, a strain of penicillium italicum, and a strain of geotrichum candidum were isolated starting from rotten oranges, and then purified and identified.

One drop of 10 μl of a stock suspension of 10⁶ spores/ml of each of 3 strains was cultivated at the surface of the Petri dishes of the Sabouraud medium with chloramphenicol containing various concentrations of the composition according to the invention. The rate of growth of the three strains is tracked daily by measuring the diameter of the colonies.

The results obtained are presented in Table 24.

TABLE 24
Change in the Diameters of the Thalli of the Three Strains that
are Cultivated in the Presence of Various Concentrations of the
Composition According to the Invention

| | Concentration | | | | | | |
|---|---|---|---|---|---|---|---|
| | | | | | 6 | | 24 |
| Strain | 0 g/l | 0.75 g/l | 1.25 g/l | 3 g/l | g/l | 12 g/l | g/l |
| _Pᵢ | 3.33 cm ± 0.03 | 0.8 cm ± 0.1 | 0 | 0 | 0 | 0 | 0 |
| _P_{d} | 3.5 cm ± 0.17 | 1 cm ± 0 | 0 | 0 | 0 | 0 | 0 |
| _G_{c} | 3.93 cm ± 0.03 | 0.66 cm ± 0.03 | 0 | 0 | 0 | 0 | 0 |

These results reveal that the composition according to the invention exerts a very strong partial inhibition on the three strains studied with the 0.75 g/l concentration that corresponds to a treatment with 0.75 kg per ton of the composition. Starting from the 1.25 concentration, total inhibition is noted on the three strains studied.

These results clearly reveal that the composition according to the invention has a very high level of effectiveness against the primary fungi responsible for the rotting of citrus fruit post-harvest.

Evaluation of the Effect of the Composition According to the Invention on Cherry Tomatoes Post-Harvest

Two batches of 120 cherry tomatoes of the Angel D1A1 variety were treated with two different preparations:
Batch 1: Containing 120 tomatoes washed with a paste containing water and the composition according to the invention at a dose of 0.5 g/l.
Batch 2: Containing 120 tomatoes washed with a paste containing chlorine.

After washing, each batch was divided into two sub-batches of 60 cherry tomatoes:
A sub-batch a: 60 tomatoes from batch 1, stored at 25° C.
A sub-batch b: 60 tomatoes from batch 2, stored at 25° C.
A sub-batch c: 60 tomatoes from batch 1, stored at 8° C.
A sub-batch d: 60 tomatoes from batch 1, stored at 8° C.

The tomatoes of each sub-batch were tracked daily, and the percentage of the dehydrated tomatoes was noted.

The results are presented in Table 25.

TABLE 25
Percentage of Rotten Cherry Tomatoes in the Batch that is Washed with the
Composition of the Invention, in Comparison with the Batch that is Washed
with Chlorine, Depending on Storage Conditions

| | 4 d | 6 d | 8 d | 10 d | 12 d | 14 d | 16 d |
|---|---|---|---|---|---|---|---|
| Sub- | 5.27% | 10.53% | 21% | 22% | 33.40% | 38.89% | 44.44% |
| Batch a | | | | | | | |
| Stored at | | | | | | | |
| 25° C. | | | | | | | |
| Sub- | 9.55% | 20.58% | 42.9% | 47.9% | 50% | 57.15% | 64.28% |
| Batch b | | | | | | | |
| Stored at | | | | | | | |
| 25° C. | | | | | | | |
| Sub- | 0% | 5% | 7.10% | 11% | 20.42% | 26.23% | 35.71% |
| Batch c | | | | | | | |
| Stored at | | | | | | | |
| 8° C. | | | | | | | |
| Sub- | 2% | 6.67% | 10% | 15% | 30% | 37.68% | 50% |
| Batch | | | | | | | |
| Stored at | | | | | | | |
| 8° C. | | | | | | | |

The results obtained reveal that the percentage of dehydrated tomatoes in sub-batches preserved at a temperature of 25° C. is higher in comparison with the sub-batches preserved at a temperature of 8° C. The results also reveal that the batch treated with the composition according to the invention has a smaller percentage of dehydrated tomatoes than the number of dehydrated tomatoes in the batch treated with chlorine.

Another test was carried out. Four batches of 20 cherry tomatoes of the Angel D1A1 variety were treated with three different preparations:
Batch 1: Containing 20 cherry tomatoes washed with the composition according to the invention at a dose of 0.5 g/l.
Batch 2: Containing 20 cherry tomatoes washed with chlorine.
Batch 3: Containing 20 cherry tomatoes washed with just water (control).

After treatment, the tomatoes from each batch were damaged at the stem with a fine syringe (22 G×1/4″) and preserved at a temperature of 27° C.

The tomatoes were monitored daily, and the percentage of tomatoes that have stem rot was noted.

The results are presented in Table 26.

TABLE 26
Percentage of Cherry Tomatoes that Have Stem Rot Depending
on the Treatment Used

| | 1 d | 2 d | 3 d | 4 d | 5 d | 6 d |
|---|---|---|---|---|---|---|
| Tomatoes | 15% | 20% | 50% | 60% | 100% | 100% |
| Washed | | | | | | |
| with Just | | | | | | |
| Water | | | | | | |
| Tomatoes | 6% | 15% | 22% | 35% | 47% | 60% |
| Washed | | | | | | |
| with | | | | | | |
| Chlorine | | | | | | |
| Tomatoes | 0% | 9% | 12.50% | 12.50% | 28.60% | 40% |
| Washed | | | | | | |
| with the | | | | | | |
| Composition | | | | | | |
| According | | | | | | |
| to the | | | | | | |
| Invention | | | | | | |

The results reveal a very significant reduction in the percentage of rotten tomatoes in the fruits washed with the composition of the invention in relation to the control fruits washed with water. This reduction is also noteworthy in relation to the fruits washed with chlorine.

1. Composition in a form that is solid, stable, and dispersible in water, appearing in the form of powder, pellets, or tablets, comprising:
at least one aromatic alcohol or at least one mixture containing at least one aromatic alcohol,
at least one emulsifying agent and/or one oil,
at least one texturing agent, and
at least one effervescent acid-base pair.

2. Composition according to claim 1, wherein:
the aromatic alcohol(s) or the mixture(s) of aromatic alcohol(s) represent(s) between 0.1 and 25% by weight of the composition,
the emulsifying agent(s) and/or the oil(s) represent(s) between 0.01 and 2% by weight of the composition,
the texturing agent(s) represent(s) between 5 and 12% by weight of the composition,
the effervescent acid-base pair(s) represent(s) between 50 and 94.89% by weight of the composition.

3. Composition according to claim 1, wherein the composition consists exclusively of:
at least one aromatic alcohol or at least one mixture containing at least one aromatic alcohol,
at least one emulsifying agent and/or one oil,
at least one texturing agent, and
at least one effervescent acid-base pair.

4. Composition according to claim 3, wherein:
the aromatic alcohol(s) or the mixture(s) of aromatic alcohol(s) represent(s) between 0.1 and 25% by weight of the composition,
the emulsifying agent(s) and/or the oil(s) represent(s) between 0.01 and 2% by weight of the composition,
the texturing agent(s) represent(s) between 5 and 12% by weight of the composition,
the effervescent acid-base pair(s) represent(s) between 61 and 94.89% by weight of the composition.

5. Composition according to claim 1, wherein in an effervescent pair, the acid of the effervescent pair represents between 20 and 25% by weight of the composition, and the base of the effervescent pair represents between 60 and 74.89% by weight of the composition.

6. Composition according to one claim 1, wherein the aromatic alcohol(s) is (are) mono-, di- or sesquiterpenic.

7. Composition according to claim 1, wherein the aromatic alcohol(s) is (are) selected from among thymol, eugenol, carvacrol.

8. Composition according to claim 1, wherein the aromatic alcohol(s) is (are) (a) natural or synthetic aromatic alcohol (s).

9. Composition according to claim 1, wherein the emulsifying agent(s) is (are) selected from among soy lecithin, cellulose, pectin, and glycerol.

10. Composition according to claim 1, wherein the oil(s) is (are) selected from among vegetable oils and mineral oils.

11. Composition according to claim 1, wherein the texturing agent(s) is (are) selected from among locust bean gum, guar gum, and cassia gum.

12. Composition according to claim 1, wherein the effervescent acid-base pair(s) is (are) selected from among the following pairs: sodium bicarbonate-citric acid, calcium bicarbonate-citric acid, potassium bicarbonate-citric acid, sodium bicarbonate-tartaric acid, calcium bicarbonate-tartaric acid, potassium bicarbonate-tartaric acid, sodium bicarbonate-maleic acid, potassium bicarbonate-maleic acid, sodium bicarbonate-ascorbic acid, calcium bicarbonate-ascorbic acid, and potassium bicarbonate-ascorbic acid.

13. A phytosanitary product for preventing and/or combatting the illnesses of plants caused by fungi, bacteria, viruses, nematodes, and/or pests, comprising the composition of claim 1.

14. A method for preventing and/or combatting illnesses of fruits, vegetables, and/or flowers post-harvest, comprising applying an effective amount of the phytosanitary product of claim 13.

15. The method of claim 14, wherein the method is used for irrigation water or in leaf spraying.

16. The method of claim 14, wherein the illnesses of plants caused by bacteria are selected from among the soft rot caused by the Erwinia species, the bacterial canker caused by the Pseudomonas species, or the crown gall caused by the Agrobacterium species.

17. The method of claim 14, wherein the illnesses of plants caused by fungi are selected from among the Fusarium wilt caused by the Fusarium species, the mildew caused by the Phytophtora species, the powdery mildew caused by the Podosphaera and Oidium species, the early blight caused by the Alternaria species, the sooty mold caused by the Alternaria and Cladosporium species, or the gray rot caused by the Botrytis species.

18. The method of claim 14, wherein the pests are selected from among aphids, gnats, soil mites, or budworms.

19. The method of claim 14, performed for preventing and/or combatting the citrus fruit rot caused by the species Penicillium and Geotrichum, or for preventing and/or combatting the fungal deterioration of dates caused by the Aspergillus species.

20. The method of claim 14, performed to provide an antifungal treatment during the coating of seeds.

21. The method of claim 20 in which the seeds are selected from among wheat, barley, lentils, chickpeas, and beans.

22. A method for performing any of the following, comprising applying an effective amount of the composition according to claim 1:
increasing the vase life of cut flowers by applying the composition as a phytosanitary product, and/or
cleaning and eliminating the traces of pesticides and microbes contained in fruits and vegetables by applying the composition as a phytosanitary product, and/or
stimulating the growth of plants, and/or
decontaminating agricultural farming substrates above ground and in the ground and/or sanitizing the ambient air, and/or
cleaning and preventing the formation of limestone.

23. The method according to claim 22, wherein the composition is applied as a phytosanitary product for cleaning and eliminating the traces of pesticides and microbes contained in the fruits and vegetables, for decontaminating grapes, apricots, peppers, and tomatoes before the drying.

24. The method according to claim 22, wherein the composition is applied for stimulating the rhizogenesis in plants.

25. A method of performing any of the following, comprising applying an effective amount of the composition according to claim 1 in animal-breeding buildings for:
Sanitizing ambient air of the buildings, and/or
Decontaminating drinking water, and/or
Cleaning surfaces of the buildings.

26. A method of performing any of the following, comprising applying an effective amount of the composition according to claim 1:
eliminating the biofilms and preventing their formation, and/or
preventing and treating avian flu in animal-breeding buildings, and/or
decontaminating animal carcasses in slaughterhouses or decontaminating poultry eggs before placing them in incubators by using the composition as a bath, and/or
disinfecting reusable medical devices, and/or
Disinfecting teeth and gums by using the composition as a mouthwash or using the composition as a disinfectant product for hands.
